(19) United States
(12) Patent Application Publication
(10) Pub. No.: US 2025/0186510 A1
(43) Pub. Date: Jun. 12, 2025

(54) BACTERIAL COMPOSITION FOR USE IN TREATMENT OF SUBSTANCE USE DISORDER

(71) Applicant: Bar-Ilan University, Ramat Gan (IL)

(72) Inventors: Gal YADID, Shoham (IL); Tzofnat BARELI, Hod Hasharon (IL)

(21) Appl. No.: 18/845,060

(22) PCT Filed: Mar. 8, 2023
(86) PCT No.: PCT/IL2023/050241

§ 371 Date: Sep. 9, 2024

Related U.S. Application Data
(60) Provisional application No. 63/317,551, filed on Mar. 8, 2022.

(51) Int. Cl. A61K 35/74; A61K 9/00; A61K 31/5685; A61P 25/30

(52) U.S. Cl. CPC A61K 35/74; A61K 9/0053; A61K 31/5685; A61P 25/30

(57) ABSTRACT

The present invention is directed to a method of treating a substance-use disorder (SUD) in a subject in need thereof, including administering to the subject a therapeutically effective amount of a pharmaceutical composition including bacteria, wherein at least 50% of the bacteria belong to a bacterial family being any one of: Ruminococcaceae, Rikenellaceae, and both, such as R. gnavus.

### REFERENCE TO AN ELECTRONIC SEQUENCE LISTING

The contents of the electronic sequence listing (BIU-P-044-PCT.xml; size: 6,152 bytes; and date of creation: Mar. 8, 2023) is herein incorporated by reference in its entirety.

### CROSS-REFERENCE TO RELATED APPLICATIONS

This application claims the benefit of priority of U.S. Provisional Patent Application No. 63/317,551, titled “COCAINE ALTERS THE GUT MICROBIOTA: REVERSAL BY TREATMENT WITH A SPECIFIC BACTERIA”, filed Mar. 8, 2022, the contents of which are incorporated herein by reference in their entirety.

### FIELD OF THE INVENTION

The present invention relates to bacterial composition, and use of same, such as in a method for treatment of a substance use disorder.

### BACKGROUND

Substance use disorders (SUDs) represent a dire public health problem. Cocaine is a major addictive substance that harms millions of people worldwide every year and costs the Office of the US Surgeon General billions of dollars annually. While physical addiction to cocaine may not develop as rapidly as with other narcotic drugs, like heroin, the psychological addiction arises very quickly. Repeated exposure to cocaine may lead to compulsive behavior and severe withdrawal symptoms. Nevertheless, there is still a lack of effective and safe medication to aid in the treatment of cocaine addiction.

The human body is colonized by large numbers of microorganisms, including bacteria, fungi, and viruses, together termed “the human microbiota”. Their abundance is influenced by several factors such as diet, environment, and host genetics. The composition of the core gut microbiota, throughout adulthood, is relatively stable, though perturbations, such as environmental stressors or changes in diet, may lead to fluctuations in species composition that may influence health or disease risk. The human gut microbiota has numerous positive benefits for host health and physiology. These benefits include nutrient secretion, vitamin synthesis, biodegradation of complex dietary sugars and glycans, modulation of intestinal and immune cell proliferation and development, and protection against infection by opportunistic bacteria.

In recent years, intestinal bacteria emerged as a potential regulator in the pathogenesis of psychiatric disorders such as depression and anxiety. However, current data indicate lack of clarity regarding interactions between the microbiota and SUDs, such as cocaine addiction. A study that compared between low- and high-responders to cocaine revealed relative abundance of Akkermansia muciniphila among future low responders. Moreover, it was recently demonstrated that the endogenous neuro-steroid dehydroepiandrosterone (DHEA) has a role in cocaine-seeking behavior in a self-administration model in rats, where pretreatment with DHEA attenuated cocaine-seeking behavior and elevated the levels of dopamine and serotonin in several brain regions relevant to cocaine addiction.

There is still a great need for an effective and safe probiotic medication, such as including bacteria belonging to Ruminococcaceae, Rikenellaceae, or both, for the treatment of SUD, including a cocaine addiction.

### SUMMARY

The present invention, in some embodiments, is based, at least in part, on a model of substance use disorder (SUD), such as cocaine-trained rats, wherein the bacterial families Ruminococcaceae and Rikenellaceae, were shown to be induced at the end of the extinction phase (on day 22), compared to the first day of extinction phase (day 13, FIGS. 1G-1H), thus, indicating a possible therapeutic role for these gut bacterial families in substance use withdrawal. The present invention, in some embodiments, is based, at least in part, on the surprising finding that administration (e.g., oral) of Ruminococcaceae bacteria, such as the specie R. gnavous, attenuated cocaine craving (FIG. 3C). This surprising finding was accompanied by an increase of ˜50% in the abundance of R. gnavous in the gut (FIG. 4C). Surprisingly and unexpectedly, in vitro assay, examining the direct effect of DHEA on the gut microbiome, indicated that incubation of feces from naïve rats with 500 μM DHEA, increased 16 bacterial features, however none of them belonged to R. gnavus (FIG. 4F), indicating that DHEA and R. gnavus have distinct mechanisms for ameliorating SUDs, such as cocaine. Therefore, in view of the facts that DHEA is a compound known to be effective in treatment of SUD or ameliorating at least one symptom associated therewith, and the similar effect induced by probiotic administration as disclosed herein, it is suggested to treat SUD or ameliorate at least one symptom associated therewith, with a composition comprising bacteria belonging to Ruminococcaceae, Rikenellaceae, or both.

According to one aspect, there is provided a method of treating a substance-use disorder (SUD) in a subject in need thereof, the method comprising administering to the subject a therapeutically effective amount of a pharmaceutical composition comprising bacteria, wherein at least 50% of the bacteria belong to a bacterial family being any one of: Ruminococcaceae, Rikenellaceae, and both, thereby treating SUD in the subject.

According to another aspect, there is provided a method of treating SUD in a subject in need thereof, the method comprising administering to the subject a therapeutically effective amount of a pharmaceutical composition comprising Ruminococcus gnavus bacteria, thereby treating SUD in the subject.

According to another aspect, there is provided a pharmaceutical composition comprising bacteria, wherein at least 50% of the bacteria belong to a bacterial family being any one of: Ruminococcaceae, Rikenellaceae, and both, for use in treatment of SUD in a subject in need thereof.

According to another aspect, there is provided a pharmaceutical composition comprising Ruminococcus gnavus bacteria for use in treatment of SUD, in a subject in need thereof.

In some embodiments, the bacterial family is Ruminococcaceae.

In some embodiments, the bacteria belong to the genus Ruminococcus.

In some embodiments, the bacteria belonging to the genus Ruminococcus is Ruminococcus gnavus.

In some embodiments, the subject is afflicted with a severe form of the SUD.

In some embodiments, the severe form of the SUD comprises an addiction to the substance.

In some embodiments, the SUD is a stimulant-related disorder.

In some embodiments, the stimulant is selected from the group consisting of: cocaine, amphetamine, cathinone, 3,4-methylenedioxymethamphetamine (MDMA), nicotine, any derivative thereof, and any combination thereof.

In some embodiments, the stimulant-related disorder is a cocaine-related disorder.

In some embodiments, the subject is addicted to cocaine or rehabilitating therefrom.

In some embodiments, the effective amount of the pharmaceutical composition comprises the bacteria in an amount of at least 1×10⁴ colony forming unit (CFU).

In some embodiments, the administering comprises orally administering.

In some embodiments, the administering comprises at least once a week administering.

In some embodiments, treating comprises lowering craving of the subject to the substance.

In some embodiments, the treating comprises increasing an amount of the bacteria belonging to a bacterial family being any one of: Ruminococcaceae, Rikenellaceae, and both by at least 50% in the subject, compared to a control.

In some embodiments, the increased amount of the bacteria belonging to a bacterial family being any one of: Ruminococcaceae, Rikenellaceae, and both, is determined in a sample obtained or derived from the administered subject.

In some embodiments, the method further comprises a step preceding the administering step, comprising determining an amount of bacteria belonging to a bacterial family being any one of: Ruminococcaceae, Rikenellaceae, and both, in a sample obtained or derived from the administered subject.

In some embodiments, treating comprises increasing an amount of Ruminococcus gnavus bacteria in the subject, compared to a control.

In some embodiments, the increased amount of the Ruminococcus gnavus bacteria is determined in a sample obtained or derived from the administered subject.

In some embodiments, the method further comprises a step preceding the administering step, comprising determining an amount of said Ruminococcus gnavus bacteria in a sample obtained or derived from the administered subject.

In some embodiments, the method further comprises administering to the subject an effective amount of dehydroepiandrosterone (DHEA), a functional analogue thereof, or a composition comprising thereof.

In some embodiments, the effective amount comprises between 0.1 mg/kg (body weight) to 5 mg/kg (body weight) of any one of DHEA and the analogue thereof.

In some embodiments, administering the DHEA comprises orally administering, intravenously administering, intraperitoneal administering, or any combination thereof.

In some embodiments, the pharmaceutical composition for use as disclosed herein is formulated for oral administration.

In some embodiments, the pharmaceutical composition for use as disclosed herein further comprises DHEA.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the study of the following detailed description.

### BRIEF DESCRIPTION OF THE FIGURES

FIGS. 1A-1H include a scheme of a non-limiting study design, and graphs demonstrating microbial features of cocaine self-administration and extinction of cocaine seeking. (1A) Experimental course. Self-administration training under fixed-ratio 1. (1B) Effect of DHEA on cocaine seeking during extinction. DHEA treatment given on days 13-22 significantly reduced cocaine-seeking behavior throughout extinction and withdrawal training, as compared with saline-treated controls (Coc-Vehicle). A two-way ANOVA with repeated measures revealed a significant effect of time (F(21,462)=17.61 p<0.0001) and an interaction between treatment and time (F(42,462)=4.56 p<0.0001) and did not reveal a significant effect of treatment (F(2,462)=3.20 p=0.0603). (Bonferroni, *** p<0.001 day 13, DHEA-treated vs. Coc-Vehicle group, ###p<0.001 days 13 and 14, ##p<0.01 days 15 and 16 saline-vehicle vs. Coc-Vehicle group). Values are expressed as mean±SEM. (1C) PCOA plot of unweighted UniFrac distances (beta diversity) of rats that received cocaine after 3 days (acquisition, red n=8) and after 12 days (maintenance, blue n=7) compared to rats that received saline after 3 days (orange n=5) and after 12 days (green n=6). (1D-1E) Linear discriminant analysis (LDA) effect size (LEfSe) was used to test the differences in relative abundances of bacterial taxa between (1D) Acquisition (day 3) vs maintenance (day 12) phases in saline-trained rats. The LEfSe show the taxa that were more abundant in saline day 3 and (1E) cocaine-trained rats. The LEfSe show the taxa that were more abundant in cocaine day 12. (1F-1H) Analysis of composition of microbiomes (ANCOM) was performed to identify microbial features that differ between (1F) cocaine-trained rats vs saline-trained rats in maintenance (day 12); (1G-1H) cocaine-trained rats at the beginning extinction phase (day 13) vs the end of extinction (day 22). (1G) Unclassified species of Ruminococcaceae; (1H) Unclassified species of Rikenellaceae.

FIGS. 2A-2B include graphs demonstrating the effect of DHEA on the microbiome. (2A) PCoA plot of unweighted UniFrac distances (beta diversity) between rats trained to self-administer cocaine and then treated with DHEA in the extinction phase (day 13, red n=3; and day 22, blue n=3), rats that were trained to self-administer cocaine and then treated with saline in the extinction phase (day 13, orange n=6; and day 22, green n=7) and rats that trained to self-administer saline and then treated with saline in the extinction phase (day 13, purple n=6; and day 22, yellow n=7). The dotted ellipse highlights that DHEA treatment induces a microbiota more similar to the saline control (Saline-Vehicle). (2B) ANCOM was performed to identify microbial features that differ between the groups at the end of the extinction phase (day 22). Levels of R. gnavus rose in rats trained to self-administer cocaine and treated with DHEA.

FIGS. 3A-3D include a scheme of a non-limiting study design, and graphs showing the effect of R. gnavus on cocaine craving. (3A) Experimental course depicting a fixed ratio cocaine self-administration model. (3B) Cocaine or saline self-administration and R. gnavus supplementation at the extinction phase. Rats (n=8-9) were trained for cocaine or saline self-administration on days 1-9. R. gnavus treatment given on days 11 and 16 did not reduce cocaine-seeking behavior throughout extinction training, as compared with saline-treated controls (Coc-Vehicle). A two-way ANOVA with repeated measures did not reveal a significant effect of treatment (F(1,240)=0.30 p=0.5947) or interaction between treatment and time (F(16,240)=0.26 p=0.9984) but revealed a significant effect of time (F(16,240)=18.53 p<0.0001). (3C) Effect of R. gnavus on cocaine-primed reinstatement. Rats received a priming injection of cocaine (10 mg/kg, IP, day 18) and were placed in the self-administration chamber with no cocaine available in the relapse test. R. gnavus delivery significantly decreased active lever pressing compared to controls (Coc-Vehicle). A one-way ANOVA revealed a significant effect of treatment (F(3,20)=6.461, p=0.0031) in the relapse test (Newman-Keuls post hoc test, *p<0.05). No change was observed in active lever presses of the Saline-Vehicle group compared to Saline-R. gnavus group (p>0.05). Values are expressed as mean+SEM. (3D) Validation of the presence of R. gnavus in feces of rats following R. gnavus gavage. Rats were trained to self-administer cocaine or saline. At extinction, some rats underwent gavage application of R. gnavus (Coc-R. gnavus, Saline-R. gnavus) and counterpart controls underwent gavage application of with PBS (Coc-Vehicle). Relative expression levels of R. gnavus ATCC 29149 PblA gene were tested in the feces at day 17. A one-way ANOVA revealed a significant general effect of treatment (F(3,19)=5.868, p=0.0052). Newman-Keuls post hoc tests revealed a significant increase in Coc-R. gnavus vs Coc-Vehicle, *p<0.05. Values are expressed as mean±SEM, n=8,9 per group.

FIGS. 4A-4F include graphs showing the in vitro effect of various concentrations of cocaine and DHEA on the relative abundances of bacterial taxa in feces of naïve rats. (4A) PCoA plot of unweighted UniFrac distances (beta diversity) between test tubes containing feces of a naïve rat incubated with 100 μM cocaine concentration and those containing feces of a naïve rat incubated with PBS (cocaine 100 μM: red, n=4; PBS: blue, n=4), p=0.047. (4B-4C) Linear discriminant analysis (LDA) effect size (LEfSe) was used to test the differences in relative abundances of bacterial taxa between (4B) 100 μM cocaine and PBS and (4C) 500 μM cocaine and PBS. (4D-4E) PCoA plot of weighted UniFrac distances (beta diversity) between a tube containing feces of a naïve rat with PBS and (4D) 100 μM DHEA (DHEA 100: red, n=3; PBS: blue, n=4) p=0.036 or (4E) 500 μM DHEA (DHEA 500: red, n=4; PBS: green, n=4) p=0.02. (4F) LEfSe analysis was performed to compere between 500 μM DHEA and PBS. The LEfSe show taxa that rose in DHEA 100 μM group.

### DETAILED DESCRIPTION

According to one aspect there is provided a method of treating an addiction or ameliorating at least one symptom associated therewith in a subject in need thereof, the method comprising administering to the subject a therapeutically effective amount of a composition comprising bacteria, or a portion thereof, wherein at least 50% of the bacteria or portion thereof, belong to a bacterial family being: Ruminococcaceae, Rikenellaceae, or both.

As used herein, the term “addiction” refers to a neuropsychological disorder of the reward system in the brain (also known as “the mesolimbic system”). In some embodiments, addiction is characterized by a persistent and intense urge to engage in a certain behavior or to consume a substance. Addiction is also known to be derived from chronic exposure to high levels of an addictive stimulus (e.g., a substance or a behavior), which leads to transcriptional and epigenetic mechanisms. Addictions center around alterations in the mesolimbic dopamine pathway, (also known as “the reward circuit”), which begins in the ventral tegmental area (VTA). One known dominant shared transcriptional mechanism to addictions, is overexpression of ΔFosB, e.g., in the dopamine D1-type medium spiny neurons of the nucleus accumbens. ΔFosB overexpression is known to be associated with addictions to variety of substances (e.g., cocaine, alcohol, amphetamine, amphetamine derivatives, cannabinoid, methylphenidate, nicotine, opioid, phenylcyclidine, and propofol), as well as with behavioral addictions (e.g., food, sexual, gambling, internet, video and shopping). Certain epigenetic modifications of histone proteins in specific regions of the brain are also known to play a crucial role in the molecular basis of both substance addictions and behavioral addictions.

In some embodiments, the method disclosed herein is for treating an addiction being a substance use disorder (SUD), a behavioral addiction, or both. In some embodiments, the method disclosed herein is for treating SUD.

In some embodiments, the bacteria are living bacteria. In some embodiments, the bacteria are active bacteria. In some embodiments, the bacteria are dead bacteria. In some embodiments, the bacteria are inactivated and/or neutralized bacteria. In some embodiments, a portion of bacteria as disclosed herein, comprises a metabolite produced and/or secreted from the bacteria disclosed herein. In some embodiments, the portion of bacteria comprises a protein produced and/or secreted by the bacteria. In some embodiments, the portion of bacteria comprises a secretome of the bacteria. As used herein the term “secretome” refers to bacterial surface molecules, including, but not limited to proteins, secreted molecules, including but not limited to proteins, or both.

In some embodiments, secretome comprises bacterial: surface proteins, secreted proteins, or both.

In some embodiments, a portion of bacteria as disclosed herein, comprises a volatile organic compounds (VOC). As used herein, the terms “volatile organic compound” or “VOC” refer to an organic compound formed during or as part of metabolism or metabolic process of a bacteria as disclosed herein. In some embodiments, a portion of bacteria comprises a nucleic acid of bacteria (e.g., DNA or RNA). In some embodiments, a portion of bacteria comprises a medium wherein the bacteria were cultured. In some embodiments, any one of the bacterial cell, the metabolite, the protein, the secretome, the VOC, the nucleic acid, the medium, or any combination thereof, is derived or obtained from the bacterial family Ruminococcaceae, Rikenellaceae or a growth medium thereof. In some embodiments, any one of the bacterial cell, the metabolite, the protein, the secretome, the VOC, the nucleic acid, the medium, or any combination thereof, is derived or obtained from Ruminococcus gnavus or a growth medium wherein Ruminococcus gnavus is or was cultured. In some embodiments, the metabolite is derived from Ruminococcus gnavus (e.g., sialic acid derivative, tryptamine, indolacetate, and trimethylamine N-oxide). Methods for obtaining, determining, and/or quantifying a portion of bacteria as disclosed herein are known in the art. Non-limiting examples include bacterial cell disruption by bead beating technology, as well as subsequent determination methods, such as, but not limited to, PCR, western blot, immunoblot, mass spectrometry, etc., all of which would be known and apparent to one of ordinary skill in the art of cellular and molecular biology, as well as biochemistry.

In some embodiments, the subject is addicted to the substance disclosed herein. In some embodiments, the subject is addicted to the substance or rehabilitating therefrom. In some embodiments, the subject is afflicted with at least one withdrawal symptom derived from the substance withdrawal. In some embodiments, treating comprises diminishing substance craving by the subject. In some embodiments, treating comprises facilitating substance use cessation. In some embodiments, treating comprises decreasing self-administration of the substance.

In some embodiments, treating comprises ameliorating at least one symptom associated with SUD in the subject. In some embodiments, at least one symptom associated with SUD comprises craving of the subject to the used substance. In some embodiments, treating comprises diminishing or reducing substance craving by the subject, facilitating substance use cessation, or both. Symptoms associated with SUD, or with substance use cessation, are known in the art. Non-limiting examples include exhaustion, fatigue, anhedonia, inability to concentrate, depression, anxiety, behavioral changes (e.g., crying, delusions of persecution, mistrust, suspiciousness, combativeness), false or unusual sense of well-being, feeling of unreality, craving for the substance, all-over body aches, pain, tremors, shakiness, and chills. In some embodiments, the subject is a mammal subject. In some embodiments, the subject is a human subject.

As used herein, the term “substance use disorder (SUD)” encompasses a mental disorder that affects a subject's brain and behavior, leading to an inability to control use of a substance, such as legal or illegal drugs, alcohol, or medications. In some embodiments, SUD involves use or abuse of a psychoactive compound (e.g., alcohol, caffeine, cannabis, hallucinogen, inhalant, opioid, sedative, hypnotic, anxiolytic, stimulant, tobacco, or any combination thereof). As used herein, the term “substance” encompasses a psychoactive compound. In some embodiments, the psychoactive compound is addictive to a subject. In some embodiments a substance is any one of: a stimulant, alcohol, cannabis or a cannabinoid(s), hallucinogen, inhalant, opioid, sedative, hypnotic, anxiolytic, nicotine, tobacco, or any combination thereof. Symptoms of SUD can range from mild to severe. In some embodiments, addiction is a severe form of SUD.

A known skilled in the art would know to diagnose SUD. The category of SUD, comprising substance abuse and substance dependence, is defined in the Diagnostic and Statistical Manual of Mental Disorders 5th edition (2013, DSM-5). In some embodiments, SUD comprises a substance abuse. In some embodiments, SUD comprises a substance dependence. In some embodiments, the severity of an individual's SUD is qualified as mild, moderate, or severe based on how many of the diagnostic criteria described in DSM-5 are met. In some embodiments, SUD comprises a mild SUD. In some embodiments, SUD comprises a moderate SUD. In some embodiments, SUD comprises a severe SUD. The International Classification of Diseases 11th revision (ICD-11) divides SUD to two distinct categories: 1) a harmful pattern of substance use; and 2) a substance dependence. In some embodiments, SUD comprises a harmful pattern of substance use. In some embodiments, SUD comprises a substance dependence.

In some embodiments, the subject is afflicted with a mild SUD. In some embodiments, the subject is afflicted with a moderate SUD. In some embodiments, the subject is afflicted with a severe SUD. In some embodiments, severe SUD comprises an addiction to the substance, as disclosed herein.

In some embodiments, the method disclosed herein is for treating SUD. In some embodiments, SUD comprises or is characterized by reduced hippocampal neurogenesis levels. SUD types, in which reduced hippocampal neurogenesis levels is observed, have been described in the art, such as, but not limited to, stimulant use disorder, opioid use disorder, marijuana use disorder, nicotine use disorder, sedative use disorder, hallucinogen use disorder, alcohol use disorder, or any combination thereof.

In some embodiments, at least 50% of the bacteria of the composition belong to the bacterial family Ruminococcaceae. In some embodiments, at least 50% of the bacteria in the composition belong to the bacterial family Rikenellaceae. In some embodiments, at least 50% of the bacteria of the composition belong to the bacterial families Ruminococcaceae and Rikenellaceae. In some embodiments, at least 40%, at least 45%, at least 50%, at least 55%, at least 60%, at least 65%, at least 70%, at least 75%, at least 85%, at least 90%, at least 95%, at least 97%, or at least 99% of the bacteria in the composition, or any value and range therebetween, belong to the bacterial family selected from: Ruminococcaceae, Rikenellaceae, or both. Each possibility represents a separate embodiment of the invention. In some embodiments, 40%-100%, 45%-100%, 50%-100%, 55%-100%, 60%-100%, 65%-100%, 70%-100%, 75%-100%, 85%-100%, 90%-100%, 95%-100%, 97%-100%, or 99%-100% of the bacteria in the composition, belong to a bacterial family selected from: Ruminococcaceae, Rikenellaceae, or both. Each possibility represents a separate embodiment of the invention.

In some embodiments, the bacteria belonging to the bacterial family Rikenellaceae is of the genus Rikenella, the genus Alistipes, or both. In some embodiments, the bacteria belonging to the genus Rikenella comprises or consists of R. microfusus. In some embodiments, the bacteria belonging to the genus Alistipes comprises or consists of: A. putredinis, A. finegoldii, A. onderdonkii, A. shahii, or any combination thereof. In some embodiments, the bacteria belonging to bacterial family Rikenellaceae is anaerobic bacteria.

In some embodiments, the bacteria belonging to the bacterial family Ruminococcaceae is of the family Oscillospiraceae. In some embodiments, the bacteria belonging to the bacterial family Ruminococcaceae is an anaerobic bacterium. In some embodiments, the bacteria belonging to the bacterial family Ruminococcaceae comprises or consists of the genus Ruminiclostridium, the genus Ruminococcoides, the genus Ruminococcus, or any combination thereof.

In some embodiments, the bacteria belonging to the bacterial family Ruminococcaceae, comprises or consists of the genus Ruminococcus. In some embodiments, at least 50% of the bacteria in the composition belong to the bacterial genus Ruminococcus. In some embodiments, at least 40%, at least 45%, at least 50%, at least 55%, at least 60%, at least 65%, at least 70%, at least 75%, at least 85%, at least 90%, at least 95%, at least 97%, or at least 99% of the bacteria in the composition, or any value and range therebetween, belong to the bacterial genus Ruminococcus. Each possibility represents a separate embodiment of the invention. In some embodiments, 40%-100%, 45%-100%, 50%-100%, 55%-100%, 60%-100%, 65%-100%, 70%-100%, 75%-100%, 85%-100%, 90%-100%, 95%-100%, 97%-100%, or 99%-100% of the bacteria in the composition, belong to the bacterial genus Ruminococcus. Each possibility represents a separate embodiment of the invention.

In some embodiments, the bacteria belonging to the bacterial genus Ruminococcus is of the class Clostridia. In some embodiments the genus Ruminococcus is paraphyletic. As used herein, the bacterial genus Ruminococcus refers to a genus classified by taxonomy NCBI ID: txid1263, Ruminococcus sijpesteijn 1948, NCBI BLAST name: firmicutes. It is noted that according to recent studies, the species: Ruminococcus gnavus, Ruminococcus hansenii, Ruminococcus hydrogenotrophicus, Ruminococcus luti, Ruminococcus productus, Ruminococcus schinkii, Ruminococcus obeum, Ruminococcus gauvreauii, Ruminococcus lactaris, Ruminococcus torques and possibly other species belonging to the genus Ruminococcus, should be re-classified as part of the genus Blautia, belonging to the family Lachnospiraceae (as described in Liu C, et al. “Reclassification of Clostridium coccoides, Ruminococcus hansenii, Ruminococcus hydrogenotrophicus, Ruminococcus luti, Ruminococcus productus and Ruminococcus schinkii as Blautia coccoides gen. nov., comb. nov., Blautia hansenii comb. nov., Blautia hydrogenotrophica comb. nov., Blautia luti comb. nov., Blautia producta comb. nov., Blautia schinkii comb. nov. and description of Blautia wexlerae sp. nov., isolated from human faeces.” Int J Syst Evol Microbiol. 2008; 58:1896-1902; and in Lawson PA, Finegold S M. “Reclassification of Ruminococcus obeum as Blautia obeum comb. nov.” Int J Syst Evol Microbiol. 2015; 65:789-793, hereby incorporated by reference in their entirety). In regard to this aspect, the genus Ruminococcus disclosed herein, encompasses all current and previously classified species in the Ruminococcus genus, including those that were re-assigned as belonging to the genus Blautia.

In some embodiments, at least 50% of the bacteria in the composition is: Ruminococcus gnavus, Ruminococcus albus, Ruminococcus bromii, Ruminococcus callidus, Ruminococcus flavefaciens, Ruminococcus gauvreauii, Ruminococcus lactaris, Ruminococcus obeum, Ruminococcus torques, Ruminococcus champanellensis, Ruminococcus sp. SR1/5, Ruminococcus bicirculans, Ruminococcus hansenii, Ruminococcus hydrogenotrophicus, Ruminococcus luti, Ruminococcus productus, Ruminococcus schinkii, Ruminococcus obeum, Ruminococcus gauvreauii, Ruminococcus lactaris, Ruminococcus torques,, or any combination thereof.

In some embodiments, at least 50% of the bacteria in the composition, belong to the bacterial family Lachnospiraceae. In some embodiments, at least 50% of the bacteria in the composition, belong to the bacterial genus Blautia. In some embodiments, the bacteria belonging to the bacterial genus Blautia, is selected from: Blautia acetigignens, Blautia ammoniilytica, Blautia argi, Blautia caecimuris, Blautia caecimuris, Blautia celeris, Blautia coccoides, Blautia faecicola, Blautia faecis, Blautia glucerasea, Blautia hansenii, Blautia hominis, Blautia hydrogenotrophica, Blautia intestinalis, Blautia liquoris, Blautia luti, Blautia obeum, Blautia producta, Blautia schinkii, Blautia stercoris, Blautia wexlerae, or any combination thereof. In some embodiments, at least 40%, at least 45%, at least 50%, at least 55%, at least 60%, at least 65%, at least 70%, at least 75%, at least 85%, at least 90%, at least 95%, at least 97%, or at least 99% of the bacteria in the composition, or any value and range therebetween, belong to the bacterial genus Blautia. Each possibility represents a separate embodiment of the invention. In some embodiments, 40%-100%, 45%-100%, 50%-100%, 55%-100%, 60%-100%, 65%-100%, 70%-100%, 75%-100%, 85%-100%, 90%-100%, 95%-100%, 97%-100%, or 99%-100% of the bacteria in the composition, belong to the bacterial genus Blautia. Each possibility represents a separate embodiment of the invention.

In some embodiments at least 50% of the bacteria in the composition is Ruminococcus gnavus (taxonomy NCBI ID: txid1263106, Ruminococcus gnavus CAG: 126). In some embodiments, at least 40%, at least 45%, at least 50%, at least 55%, at least 60%, at least 65%, at least 70%, at least 75%, at least 85%, at least 90%, at least 95%, at least 97%, or at least 99% of the bacteria in the composition, or any value and range therebetween, is Ruminococcus gnavus. Each possibility represents a separate embodiment of the invention. In some embodiments, 40%-100%, 45%-100%, 50%-100%, 55%-100%, 60%-100%, 65%-100%, 70%-100%, 75%-100%, 85%-100%, 90%-100%, 95%-100%, 97%-100%, or 99%-100% of the bacteria in the composition, is Ruminococcus gnavus. Each possibility represents a separate embodiment of the invention.

In some embodiments, the method comprises administering to the subject a therapeutically effective amount of a composition comprising Ruminococcus gnavus. In some embodiments, the method comprises administering to the subject a therapeutically effective amount of a composition comprising an active ingredient and a carrier, wherein the active ingredient consists essentially of a bacteria. In some embodiments, the bacteria consist essentially of Ruminococcus gnavus. In some embodiments, the bacteria consists of Ruminococcus gnavus.

The term “consists essentially of” denotes that a given compound or substance constitutes the vast majority of the active ingredient's portion or fraction of the composition.

In some embodiments, consists essentially of means that Ruminococcus gnavus constitutes at least 95%, at least 98%, at least 99%, or at least 99.9% by weight, by colony forming unit (CFU), cell number, or any combination thereof, of the bacteria of the composition, or any value and range therebetween. Each possibility represents a separate embodiment of the invention.

In some embodiments, consists essentially of means that Ruminococcus gnavus constitutes at least 95%, at least 98%, at least 99%, or at least 99.9% by weight, by colony forming unit (CFU), cell number, or any combination thereof, of the active ingredient(s) of the composition, or any value and range therebetween. Each possibility represents a separate embodiment of the invention.

Methods of determining, characterizing, and/or quantifying any one of: bacterial family, bacterial genus, bacterial specie, or any combination thereof, in a gut microbiome are known in the art. Non-limiting examples include sequencing a gene, and/or genes coding for 16S ribosomal RNA (16S rRNA), an abundantly used method in reconstructing phylogenies. As used herein, the term “gut microbiome” is well known in the art and refers to any bacteria in a gastrointestinal tract of a subject.

In some embodiments, the substance disclosed herein is selected from: alcohol, cannabis or a cannabinoid(s), opioid, stimulant (e.g., cocaine, nicotine, or amphetamines), benzodiazepine, barbiturate, or any combination thereof. In some embodiments, SUD comprises at least one disorder selected from: stimulant use disorder, opioid use disorder, marijuana use disorder, nicotine use disorder, sedative use disorder, hallucinogen use disorder, alcohol use disorder, or any combination thereof. In some embodiments, an opioid comprises heroin, a synthetic opioid (e.g., fentanyl), a prescription pain reliever (e.g., oxycodone, hydrocodone, codeine, and morphine), or any combination thereof. In some embodiments, a sedative substance comprises benzodiazepine (e.g., diazepam, clonazepam, and alprazolam), non-benzodiazepine sleep medication, (e.g., zolpidem, eszopiclone, and zaleplon), barbiturate (e.g., mephobarbital, phenobarbital, and pentobarbital sodium), or any combination thereof. In some embodiments, a hallucinogen is chemically synthesized, (e.g., lysergic acid diethylamide, or LSD). In some embodiments, a hallucinogen is a naturally occurring substance (e.g., psilocybin mushroom and peyote).

In some embodiments, the SUD disclosed herein comprises or is a stimulant-related disorder. As used herein, the terms “stimulant-related disorder” and “stimulant use disorder” are interchangeable. The terms “stimulant” and “stimulant substance” are herein used interchangeably and encompass a compound that is known to enhance the activity of the central and/or peripheral nervous systems. Stimulant substances are well known in the art. In some embodiments, a stimulant effect includes increased: alertness, awareness, wakefulness, endurance, productivity, motivation, arousal, locomotion, heart rate, blood pressure, diminished requirement for food and sleep, or any combination thereof. In some embodiments, the stimulant is selected from: cocaine, amphetamine, cathinone, 3,4-methylenedioxymethamphetamine (MDMA), nicotine, any derivative thereof, or any combination thereof.

In some embodiments, the stimulant-related disorder comprises a cocaine-related disorder. In some embodiments, the stimulant comprises cocaine (e.g., benzoylmethylecgonine), or a derivative or analogue thereof. In some embodiments, cocaine comprises native cocaine. In some embodiments, cocaine comprises erythroxylum coca. In some embodiments, cocaine comprises erythroxylum novogranatense. In some embodiments, cocaine comprises synthetic derivative or analogue of cocaine.

The terms “derivative” and “analogue” are herein used interchangeably and include any analogue of the addictive substance disclosed herein, having identical active site to the substance, as long as it has or maintains at least 70%, 80%, 90% or 95% of the psycho-activity of the substance, or any value and range therebetween. Each possibility represents a separate embodiment of the invention. As used herein, the term “psycho-activity” of a substance, refers to an effect or activity in the brain of a subject, comprising a mammal subject. The psycho-activity of a stimulant (e.g., cocaine) is well described in the art. In some embodiments, a psycho-activity of a stimulant comprises binding of the stimulant to a dopamine transporter (DAT) in a synaptic cleft, increasing dopamine concentration in the synaptic cleft, or both.

Derivatives or analogues of cocaine are known in the art. Non-limiting examples for such synthetic psychoactive derivatives include a stereoisomer of cocaine, 3β-phenyl ring substituted analogue, 2β-substituted analogue, N-modified analogue of cocaine, 3β-carbamoyl analogue, 3β-alkyl-3-benzyl tropane, 6/7-substituted cocaine, 6-alkyl-3-benzyl tropane, and piperidine homolog of cocaine.

In some embodiments, the stimulant comprises amphetamine or derivative or analogue thereof. Derivatives and/or analogues of amphetamine are common and would be apparent to one of ordinary skill in the art, non-limiting examples of which include: levoamphetamine, dextroamphetamine, lisdexamphetamine, methamphetamine, and ephedrine.

In some embodiments, the stimulant comprises cathinone (e.g., benzoylethanamine, or β-keto-amphetamine) or derivative or analogue thereof. In some embodiments, cathinone is extracted from a plant. In some embodiments, cathinone extraction is from catha edulis. In some embodiments, cathinone is chemically synthesized. Cathinone derivatives are known in the art, and non-limiting examples include: amfepramone, diethylpropion, mephedrone (4-methylmethcathinone, 4-MMC), methylone (βk-MDMA, 3,4-methylenedioxy-N-methylcathinone), methcathinone (ephedrone), MDPV (3,4-methylenedioxypyrovalerone), methcathinone (ephedrone), N,N-Dimethylcathinone (metamfepramone), N-ethylcathinone (EC), buphedrone, 4-methyl-N-ethylcathinone, mephedrone (4-MMC; M-CAT), amfepramone, bupropion, methylone (βk-MDMA), ethylone (βk-MDEA), butylone (βk-MBDB), methedrone (βk-PMMA), flephedrone (4 -FMC), 3-fluoromethcathinone (3-FMC), α-pyrrolidinopropiophenone (PPP), 4-methyl-α-pyrrolidinopropiophenone (MPPP), 4-methoxy-α-pyrrolidinopropiophenone (MOPPP), 4-methyl-α-pyrrolidino-hexanophenone (MPHP), pyrovalerone, 4-methyl-α-pyrrolidino-butyrophenone (MPBP), 4-methyl-α-pyrrolidino-α-methylpropiophenone, 3,4-methylenedioxy-α-pyrrolidinopropiophenone (MDPPP), 3,4-methylenedioxypyrovalerone (MDPV).

In some embodiments, the stimulant comprises a hallucinogen substance. In some embodiments, the hallucinogen substance comprises 3,4-methylenedioxymethamphetamine (MDMA), or analogue or derivative thereof. Analogues or derivatives of MDMA are known in the art, and non-limiting examples include empathogen (e.g., substituted methylenedioxyphenethylamine (MDxx), benzofuran, miscellaneous polycyclic phenethylamine, and tryptamine. In some embodiments, MDMA analogue comprises 3,4-methylenedioxyamphetamine (MDA), or its N-ethyl derivative; 3,4-methylenedioxy-N-ethylamphetamine (MDE).

The term “therapeutically effective amount” refers to an amount of a drug or compound (e.g., bacteria as disclosed herein) that is effective to treat a disease or disorder in a subject, such as a mammal subject. The term “a therapeutically effective amount” refers to an amount effective, at dosages and for periods of time necessary, to achieve the desired therapeutic or prophylactic result. The exact dosage form and regimen would be determined by the physician according to the patient's condition.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, an effective amount of the composition is an amount of at least 1×10⁴ colony forming unit (CFU) of the bacteria disclosed herein. In some embodiments, the composition comprises the bacteria in an amount of at least 1×10⁴ CFU, at least 1×10⁵ CFU, at least 1×10⁶ CFU, at least 1×10⁷ CFU, at least 1×10⁸ CFU, or any value and range therebetween. Each possibility represents a separate embodiment of the invention. In some embodiments, the composition comprises the bacteria disclosed herein in an amount ranging from 1×10⁴ CFU to 1×10¹⁰ CFU, from 1×10⁵ CFU to 1×10¹⁰ CFU, from 1×10⁶ CFU to 1×10¹⁰ CFU, from 1×10⁷ CFU to 1×10¹⁰ CFU, from 1×10⁸ CFU to 1×10¹⁰ CFU, from 1×10⁴ CFU to 1×10⁹ CFU, from 1×10⁵ CFU to 1×10⁹ CFU, from 1×10⁶ CFU to 1×10⁹ CFU, from 1×10⁷ CFU to 1×10⁹ CFU, or from 1×10⁸ CFU to 1×10⁹ CFU. Each possibility represents a separate embodiment of the invention.

The route of administration of the composition will depend on the disease or condition to be treated. Suitable routes of administration include, but are not limited to, oral administration, parenteral injections, e.g., intradermal, intravenous, intramuscular, intralesional, subcutaneous, intrathecal, and any other mode of injection as known in the art. Although the bioavailability of the active ingredients to be administered by other routes can be lower than when administered via parenteral injection, by using appropriate compositions it is envisaged that it will be possible to administer the compositions of the invention via transdermal, oral, rectal, vaginal, topical, nasal, inhalation and ocular modes of treatment. In addition, it may be desirable to introduce the pharmaceutical composition of the invention by any suitable route, including intraventricular and intrathecal injection. In some embodiments, administering comprises orally administering. In some embodiments, administering comprises single administration. In some embodiments, administering comprises multiple administrations.

In some embodiments, administering comprises at least once a week administering. In some embodiments, administering comprises at least once administering every 14 days, every 13 days, every 12 days, every 11 days, every 10 days, every 9 days, every 8 days, every 7 days, every 6 days, every 5 days, every 4 days, every 3 days, every 2 days, every 1 day, or any value and range therebetween. Each possibility represents a separate embodiment of the invention. In some embodiments, administering comprises once administering every 14 days, every 13 days, every 12 days, every 11 days, every 10 days, every 9 days, every 8 days, every 7 days, every 6 days, every 5 days, every 4 days, every 3 days, every 2 days, or every 1 day. Each possibility represents a separate embodiment of the invention. In some embodiments, administration comprises 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 administrations, every 14 days. Each possibility represents a separate embodiment of the invention.

In some embodiments, treating comprises increasing an amount of the bacteria disclosed herein by at least 50% in the subject disclosed herein, compared to a control subject. In some embodiments, a control subject comprises a subject afflicted with SUD, as disclosed herein, not being administered with the bacterial composition disclosed herein. In some embodiments, a control subject comprises a subject afflicted with SUD, prior administration with the bacterial composition disclosed herein. In some embodiments, treating comprises increasing an amount of the bacteria belonging to a bacterial family being any one of: Ruminocccaceae, Rikenellaceae, and both by at least 50% in the subject, compared to a control, as disclosed herein. In some embodiments, treating comprises increasing an amount of the bacteria belonging the bacterial family Ruminocccaceae by at least 50% in the subject, compared to a control, as disclosed herein. In some embodiments, treating comprises increasing an amount of the bacteria being Ruminococcus gnavus by at least 50% in the subject, compared to a control, as disclosed herein.

In some embodiments, increasing or increase comprises an increase of at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, or any value and range therebetween. Each possibility represents a separate embodiment of the invention. In some embodiments, increasing or increase comprises an increase of between 10% and 200%, between 10% and 150%, between 20% and 150%, between 30% and 150%, between 40% and 150%, between 50% and 150%, between 60% and 150%, between 70% and 150%, between 80% and 150%, between 90% and 150%, between 10% and 140%, between 20% and 140%, between 30% and 140%, between 40% and 140%, between 50% and 140%, between 60% and 140%, between 70% and 140%, between 80% and 140%, between 90% and 140%, between 10% and 130%, between 20% and 130%, between 30% and 130%, between 40% and 130%, between 50% and 130%, between 60% and 130%, between 70% and 130%, between 80% and 130%, between 90% and 130%, between 10% and 120%, between 20% and 120%, between 30% and 120%, between 40% and 120%, between 50% and 120%, between 60% and 120%, between 70% and 120%, between 80% and 120%, between 90% and 120%, between 10% and 110%, between 20% and 110%, between 30% and 110%, between 40% and 110%, between 50% and 110%, between 60% and 110%, between 70% and 110%, between 80% and 110%, between 90% and 110%. Each possibility represents a separate embodiment of the invention.

Methods for determining the amount or abundance of bacteria are known in the art (e.g., extraction of DNA, and polymerase chain reaction of a specific bacterial gene, or of the 16S subunit of the rRNA). In one embodiment, the amount of Rominococcus gnavus is determined by amplification of the ATCC 29149 PblA gene.

In some embodiments, increased amount or abundance of bacteria disclosed herein is determined in a sample obtained or derived from the administered subject. In some embodiments, the sample comprises bacteria obtained from a gastrointestinal tract of the subject. In some embodiments, the sample comprises a biopsy collected or obtained from the gastrointestinal tract of the subject. In some embodiments, the sample comprises stool obtained from the subject.

In some embodiments, the method further comprises a determining step, comprising determining an amount of a bacteria belonging to a bacterial family being any one of: Ruminococcaceae, Rikenellaceae, and both. In some embodiments, the determining step, comprises determining an amount of a bacteria belonging to a bacterial family belonging to the bacterial family Ruminococcaceae. In some embodiments, the determining step, comprises determining an amount of a bacteria belonging to a bacterial family belonging to the bacterial family Blautia. In some embodiments, the determining step, comprises determining an amount of a bacteria being Ruminococcus gnavus. In some embodiments, determining is in a sample obtained or derived from the subject, as disclosed herein.

In some embodiments, amount of a bacterial family, specie, or both, as disclosed herein, below a predetermined threshold, indicates the subject is suitable for treatment with the bacterial composition, as disclosed herein.

In some embodiments, the determining step is performed before the administering step (e.g., preceding). In some embodiments, the method further comprises a determining step being performed after the administering step (e.g., proceeding). In some embodiments, the method comprises at least a first determining step being performed before the administering step and at least a second determining step being performed after the administering step.

In some embodiments, there is provided a method for selecting a subject being suitable for treatment with the bacterial composition disclosed herein, comprising the steps of: (a) determining an amount of the bacteria belonging to a bacterial family being any one of: Ruminococcaceae, Rikenellaceae, and both, wherein an amount of the bacteria being below a predetermined threshold indicates that the subject is suitable for treatment with the bacterial composition, and (b) administering to a subject determined to be suitable for treatment according to step (a) a therapeutically effective amount of the bacterial composition disclosed herein.

In some embodiments, there is provided a method for selecting a subject being suitable for treatment with the bacterial composition disclosed herein, comprising the steps of: (a) determining an amount of a bacteria being Ruminococcus gnavus derived or obtained from the subject, wherein an amount of the bacteria being below a predetermined threshold indicates that the subject is suitable for treatment with the bacterial composition, and (b) administering to a subject determined to be suitable for treatment according to step (a) a therapeutically effective amount of the bacterial composition disclosed herein.

In some embodiments, the determining step is performed in the subject or in a sample derived or obtained from the subject. In some embodiments, the sample comprises any bodily fluid, cell, tissue, biopsy, organ, or a combination thereof, derived or obtained from the subject. In some embodiments, the determining step is performed in vivo, ex vivo, or in vitro. In some embodiments, the sample comprises a stool sample. It would be clear to a person of skill in the art that in vitro, ex vivo, or both, are not performed in a subject body, e.g., in a tube or a plate.

In some embodiments, the method further comprises treating the subject disclosed herein with a behavioral therapy. As used herein, the term “behavioral therapy” refers to known cognitive-behavioral therapies that aim to help SUD patients recognize, avoid, and/or cope with situations in which they are most likely to use addictive substances. A behavioral therapy as disclosed herein may include: (i) contingency management that uses positive reinforcement such as providing rewards or privileges for remaining drugfree, for attending and participating in counseling sessions, or for taking treatment medications as prescribed, (ii) a motivational enhancement therapy that uses strategies to make the most of people's readiness to change their behavior and enter treatment, (iii) a family therapy that helps people (e.g., young people) with drug use problems, as well as their families, address influences on drug use patterns and improve overall family functioning, and (iv) a twelve-step facilitation (TSF), which is an individual therapy typically delivered in 12 weekly session to prepare people to become engaged in 12-step mutual support programs.

In some embodiments, the method disclosed herein further comprises treating a subject with a behavioral therapy, administering an anti-SUD drug, or both. In some embodiments, the method further comprises administering an anti-SUD drug. As used herein, the term “anti-SUD drug” encompasses a known medication for treatment of SUD, or a symptom associated therewith. Common medications used to treat drug addiction and withdrawal include, but are not limited to: (i) for opioid use disorder; methadone, buprenorphine, extended-release naltrexone, and lofexidine, (ii) for nicotine use disorder: nicotine replacement therapies (available as a patch, inhaler, or gum), bupropion, and varenicline, (iii) for alcohol use disorder: naltrexone, disulfiram, and acamprosate.

In some embodiments, SUD as disclosed herein comprises a nicotine use disorder. In some embodiments, anti-SUD drug comprises at least one drug selected from: nicotine replacement therapy, bupropion, varenicline, or any combination thereof. In some embodiments, SUD comprises an alcohol use disorder. In some embodiments, anti-SUD drug comprises at least one drug selected from: naltrexone, acamprosate, disulfiram, or any combination thereof. In some embodiments, SUD comprises an opioid use disorder. In some embodiments, anti-SUD drug comprises at least one drug selected from: methadone, buprenorphine, naltrexone, lofexidine, or any combination thereof. In some embodiments, anti-SUD drug comprises at least one drug selected from: a full agonist, a partial agonist, and an antagonist drug. As used herein, the term “full agonist” refers to a drug that directly binds and stimulates a receptor in the brain (e.g., methadone), the term “partial agonist” refers to a drug that directly binds a receptor in the brain, which does not stimulate the receptor to the same degree as the addictive substance (e.g., buprenorphine), and the term “antagonist” refers to a drug that binds to the receptor, which does not stimulate it, and prevents agonists from binding it (e.g., naltrexone).

In some embodiments, SUD comprises a cocaine use disorder. In some embodiments, anti-SUD drug comprises a GABAergic medication. As used herein, the term “GABAergic medication” refers to a medication that activates the GABAergic neurons. In some embodiments, GABAergic medication decreases activation in the dopaminergic reward system. In some embodiments, an anti-SUD drug comprises at least one drug selected from: propranolol, baclofen, tiagabine, topiramate, modafinil, disulfiram, the vaccine TA-CD, capable of stimulating the production of cocaine specific antibodies, or any combination thereof.

In some embodiments, anti-SUD drug comprises dehydroepiandrosterone (DHEA). In some embodiments, the method further comprises administering to the subject an effective amount of DHEA, a functional analogue thereof, or a composition comprising thereof. In some embodiments, the method comprises administering to the subject an effective amount of the bacterial composition disclosed herein and an effective amount of DHEA, a functional analogue thereof, or a composition comprising thereof.

In some embodiments, DHEA is systemically administered. In some embodiments, DHEA is orally administered. In some embodiments, DHEA is intra-peritoneal administered. In some embodiments, DHEA is intravenously administered. In some embodiments, DHEA is dermally administered. In some embodiments, DHEA is vaginally administered. In some embodiments, DHEA is rectally administered. In some embodiments, DHEA is abdominal administered. In some embodiments, DHEA is subcutaneously administered.

As used herein, the term “functional analogue of DHEA” comprises a any synthetic DHEA analogue, having identical active site to the endogenous DHEA, as long as it has or maintains at least 70%, 80%, 90% or 95% of the neurotrophin activity of endogenous DHEA, or any value and range therebetween. Each possibility represents a separate embodiment of the invention. Effective doses of DHEA, a functional analogue thereof, or a composition comprising thereof, are well known in the art and may comprise dosage in the range between 0.1 mg/kgbw (body weight) and 10 mg/kgbw (body weight).

By another aspect, there is provided a composition comprising bacteria, wherein at least 50% of the bacteria belongs to a bacterial family being any one of: Ruminococcaceae, Rikenellaceae, and both, and an acceptable carrier, for use in a treatment of a substance-use disorder, in a subject in need thereof. In some embodiments, there is provided a composition comprising bacteria, wherein at least 50% of the bacteria is Ruminococcus gnavus for use in treatment of SUD in a subject in need thereof. In some embodiments, there is provided a composition comprising bacteria for use in treatment of SUD in a subject in need thereof, wherein the bacteria consist essentially of Ruminococcus gnavus.

In some embodiments, the composition is formulated for oral administration. In some embodiments, the composition is formulated for dermal administration. In some embodiments, the composition is formulated for vaginal administration. In some embodiments, the composition is formulated for systemic administration. In some embodiments, the composition is formulated for rectal administration. In some embodiments, the composition is formulated for abdominal administration. In some embodiments, the composition is formulated for subcutaneous administration. In some embodiments, the composition is formulated for intra-peritoneal administration. In some embodiments, the composition is formulated for intravenous administration. In some embodiments, the composition is formulated for administration to a subject.

In some embodiments, the composition is a pharmaceutical composition or a nutraceutical composition. In some embodiments, the acceptable carrier is a pharmaceutically acceptable carrier or a nutraceutically acceptable carrier. In some embodiments, the composition further comprises a pharmaceutically acceptable carrier or a nutraceutically acceptable carrier.

In some embodiments, the composition, e.g., pharmaceutical or nutraceutical composition, further comprises DHEA, a functional analogue thereof, or both.

The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned.

The carrier may comprise, in total, from about 0.1% to about 99.99999% by weight of the pharmaceutical compositions presented herein.

In some embodiments, “reduce” or “reducing” is at least a: 10%, 15%, 20%, 25%, 30%, 35%, 40%, 45%, 50%, 55%, 60%, 65%, 70%, 75%, 80%, 85%, 90%, 95%, 97%, 99%, or 100% reduction, or any value and range therebetween. Each possibility represents a separate embodiment of the invention. In some embodiments, “reduce” or “reducing” is by 5-100%, 5-90%, 5-80%, 5-70%, 5-60%, 5-50%, 5-40%, 5-30%, 5-25%, 5-20%, 5-10%, 10-100%, 10-90%, 10-80%, 10-70%, 10-60%, 10-50%, 10-40%, 10-30%, 10-20%, 20-100%, 20-90%, 20-80%, 20-70%, 20-60%, 20-50%, 20-40%, 20-30%, 30-100%, 30-90%, 30-80%, 30-70%, 30-60%, 30-50%, 30-40%, 40-100%, 40-90%, 40-80%, 40-70%, 40-60%, 40-50%, 50-100%, 50-90%, 50-80%, 50-70%, 50-60%, 60-100%, 60-90%, 60-80%, 60-70%, 70-100%, 70-90%, 70-80%, 80-100%, 80-90%, or 90-100%. Each possibility represents a separate embodiment of the invention.

In some embodiments, “increase” or “increasing” is at least a: 10%, 20%, 25%, 30%, 40%, 50%, 60%, 70%, 75%, 80%, 90%, 100%, 150%, 200%, 250%, 300%, 350%, 400%, 450%, 500%, 600%, 700%, 800%, 900%, or 1,000% increase, or any value and range therebetween. Each possibility represents a separate embodiment of the invention. In some embodiments, “increase” or “increasing” is by 5-10%, 5-50%, 5-100%, 10-50%, 10-100%, 20-100%, 30-100%, 40-100%, 50-100%, 50-200%, 50-300%, 50-400%, 50-500%, 100-200%, 100-300%, 100-400%, 100-500%, 100-600%, 100-700%, 100-800%, 100-900%, or 100-1000%. Each possibility represents a separate embodiment of the invention.

In some embodiments, “reduce” or “reducing” is compared to a control. In some embodiments, “increase” or “increasing” is compared to a control. In some embodiments, the control comprises a control subject. In some embodiments, the control subject comprises the subject in need thereof, prior treatment with the method disclosed herein. In some embodiments, the control subject comprises a subject afflicted with the same disease as the subject disclosed herein, not being treated with the method disclosed herein.

The terms “reduce”, “reducing”, “inhibit” and “inhibiting” are used interchangeably.

As used herein, the terms “administering”, “administration”, and like terms refer to any method which, in sound medical practice, delivers a composition containing an active agent to a subject in such a manner as to provide a therapeutic effect. One aspect of the present subject matter provides for topical administration, oral administration, or both, of a therapeutically effective amount of a composition of the present subject matter to a patient in need thereof. Other suitable routes of administration can include parenteral, subcutaneous, intravenous, intramuscular, or intraperitoneal.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising,” indicate the inclusion of any recited integer or group of integers but not the exclusion of any other integer or group of integers.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Materials and Methods

A number of independent experiments in which rats were trained to self-administer cocaine or saline, were conducted. Behavioral and microbiota responses were assessed both in the self-administration phase as well as the extinction phase to determine the correlation of behavioral performance to gut microbiota composition. Further, a set of in vitro experiments were used to separate the direct effect from the host response to cocaine on the microbiota.

### Animals

Male Sprague-Dawley rats (250-280 g) were maintained on a reverse 12-12-hour dark-light cycle with free access to food and water. All rats were maintained on the same chow (Envigo, Teklad Global Rodent Diets, Israel) diet. All experimental procedures were approved by the Animal Care and Use Committee of Bar Ilan University, Ramat Gan and were performed in accordance with the guidelines of the National Institutes of Health, protocol number 47-06-2018.

### Intravenous Catheterization

To facilitate cocaine administration, rats were fitted with intravenous (IV) catheters. Rats were anesthetized with ketamine hydrochloride (100 mg/kg, IP) and xylazine (10 mg/kg, IP), and then implanted with intravenous silastic catheters (Dow Corning, Midland, MI, USA) in the right jugular vein. The catheter was secured to the vein with silk sutures and was passed subcutaneously to the top of the skull where it exited into a connector (a modified 22-gauge cannula; Plastics One, Roanoke, VA, USA) mounted to the skull with MX-80 screws (Small Parts, Inc., Miami Lakes, FL, USA) and dental cement (Yates & Birds, Chicago, IL, USA). During the surgery, rats were treated with Byatril (20 mg/kg) and Rimadyl (2 mg/kg) and were then allowed to recover for five days.

### Cocaine Self-Administration Training

Rats were trained to self-administer cocaine for 12 days under an FR-1 (fixed ratio 1) schedule of reinforcement, as previously described until maintenance (five consecutive days that the active lever presses were <20%). Briefly, 5 days after catheterization, rats were transferred to operant conditioning chambers (Med-Associates, Inc.; St. Albans, VT, USA) for 1 hour daily during their dark cycle. The self-administration chambers (Med-Associates, Inc.; St Albans, VT, USA) had two levers, one active and one inactive. An active lever press generated a cocaine infusion (1.5 mg/kg, 0.13 ml, 5 sec/infusion; cocaine was obtained from the National Institutes on Drug Abuse, MD, USA) through the IV catheter which was connected to an infusion pump. Throughout cocaine infusion intervals, a light located above the active lever was lit for 20 sec, 15 sec beyond the cocaine infusion period, which lasted 5 sec. During these 15-sec intervals, active lever presses were recorded, but no additional cocaine reinforcement was provided. Presses on the inactive lever did not activate the infusion pump nor the light. The number of active lever responses, infusions, and inactive lever responses were recorded. Rats were returned to their home cages at the end of the daily session.

### Extinction Training

Rats were placed in the operant conditioning chambers for daily 1-hour sessions. In this phase, no cocaine was provided upon active lever pressing, but the light cue was still activated. Active and inactive lever presses were recorded. Throughout this phase, rats received a daily intraperitoneal (IP) injection of saline or DHEA (2 mg/kg) 90 min before each extinction test session.

### Bacterial DNA Extraction, Amplification, and Sequencing

Fecal samples were collected at specific time points throughout cocaine training and extinction phases. These time points represent critical days in the addiction progress; acquisition (day 3) vs maintenance (day 12) and the first day of extinction (day 13) vs its conclusion (day 22). The samples were collected immediately after daily sessions from rats separated into home cages containing a divider. Samples were stored at −80° C. until DNA extraction. DNA was extracted using the Mobio PowerSoil DNA Extraction Kit (Mo Bio, Carlsbad, CA, USA) following a 2 minute bead beating step as described previously. The V4 region of the bacterial 16S rRNA gene was amplified by polymerase chain reaction (PCR) using the 515F (AATGATACGGCGACCACCGAGATCTACACGCT; SEQ ID NO: 1) barcoded and 806R (TATGGTAATTGTGTGYCAGCMGCCGCGGTAA; SEQ ID NO: 2) primers. For each PCR reaction, the following reagents were added: 2 μl 515F (forward, 10 μM) primer, 2 μl 806R (reverse, 10 μM) primer, 25 μl PrimeSTAR Max PCR mix (Takara, Mountain View, CA, USA), 17 μl ultra-pure water and 4 μl of sample DNA, in a total reaction volume of 50 μl. PCR reactions were carried out with 30 cycles consisting of: denaturation (95° C. for 10 sec), annealing (55° C. for 5 sec) and extension (72° C. for 20 sec), with final elongation at 72° C. (1 min). PCR products were purified using AMPure XP magnetic beads (Beckman Coulter, Indianapolis, IN, USA) and quantified using Quant-iT PicoGreen dsDNA quantitation kit (Invitrogen, Carlsbad, CA, USA), both according to the manufacturers' protocols. Samples were then pooled in equal amounts, loaded on 2% agarose E-Gel (Thermo Fisher, Waltham, MA, USA), purified, and sequenced using the Illumina MiSeq platform (Genomic Center, Azrieli Faculty of Medicine, BIU, Israel).

Incubation of Feces with Cocaine and DHEA

To understand the gut microbiota diversity emerging from direct pharmacological cocaine or DHEA exposure on the microbiota rather than potential host-mediated effects, the inventors performed an in vitro experiment on feces of naïve rats. Bacterial taxa differences in fecal samples were analyzed using the Linear discriminant analysis effect size (LEfSe) method as a response for either cocaine or DHEA (0, 50, 100 and 500 μM). Stool of naïve rats was collected into 15 ml test tubes and stored under anaerobic conditions. One pellet of stool was added to a test tube and then cocaine or DHEA was added in various concentrations, as indicated in Table 1. The stool was homogenized using a vortex and mechanical stirring. After completion of the preparation, the fecal slurry was incubated at 37° C. under anaerobic conditions. Following a week of anaerobic incubation, DNA was extracted from the slurry, and 16S rRNA gene sequencing was performed (protocols described above).

TABLE 1
Cocaine and DHEA concentrations in in vitro experiments

| Concentration of | Amount of liquid | | |
|---|---|---|---|
| cocaine or | cocaine or DHEA | Amount of | Number |
| DHEA (μM) | (μL) | PBS (μL) | of tubes |
| 50 | 250 | 4750 | 4 |
| 100 | 500 | 4500 | 4 |
| 500 | 2500 | 2500 | 3 |
| 0 | 0 | 5000 | 3 |

Treatment with R. gnavus

R. gnavus (ATCC 29149) was grown anaerobically for 3 weeks at 37° C. using tryptic soy agar supplemented with defibrinated sheep blood. Cultures were then centrifuged, washed, resuspended in phosphate buffered saline (PBS), and used immediately for oral gavage in 8-9 rats that were applied with gavage administration of PBS (vehicle) or R. gnavus twice over the course of the extinction phase. The experimental group received 1 ml of R. gnavus (˜1×10⁸ CFU/Rat and ˜1.5×10⁸ CFU/rat, respectively), while the control group received an equal volume of PBS (vehicle).

### Cocaine-Primed Reinstatement

Rats received a single injection of cocaine (10 mg/kg; IP) 24 hours after the last R. gnavus or PBS treatment and were placed in the operant chambers for 1 hour, with no cocaine dispensed. Active lever presses were monitored to assess cocaine craving.

R. gnavus DNA Extraction from Feces and Quantitative Real-Time PCR (qPCR)

Fecal samples were analyzed for R. gnavus abundance. Total DNA was extracted using a DNeasy PowerSoil Kit (QIAGEN, Germantown, MD, USA) according to the manufacturer's protocol. Purity, integrity, and concentration of the isolated DNA samples were determined by spectrophotometric absorbance at 260 nm. Real-time PCR reactions were carried out on a Step One Plus real-time PCR system (Thermo Fisher Scientific, Waltham, MA, USA) using fluorescent SYBR Green fast mix technology (qScript cDNA Synthesis; Quanta BioSciences Inc, Gaithersburg, MD, USA). Reaction protocols were as follows: 30 sec at 95° C. for enzyme activation, followed by 45 cycles of 5 sec at 95° C. and 30 sec at 60° C. Melting curve analysis was used to confirm the specificity of the amplification products. Primers (synthesized by Integrated DNA Technologies (IDT), Coralville, IA, USA) for the R. gnavus ATCC 29149 PblA gene were designed as follows: PblA: F: 5′-ACAGGGACAACGGCAAAAGA-3′ (SEQ ID NO: 3), and R: 5′-GCCATTCCAGCTACCCAGTT-3′ (SEQ ID NO: 4); and general bacteria primers: F: 5′-ACTCCTACGGGAGGCAGCAG-3′ (SEQ ID NO: 5), and R: 5′-ATTACCGCGGCTGCTGG-3′ (SEQ ID NO: 6).

### Experimental Groups Description

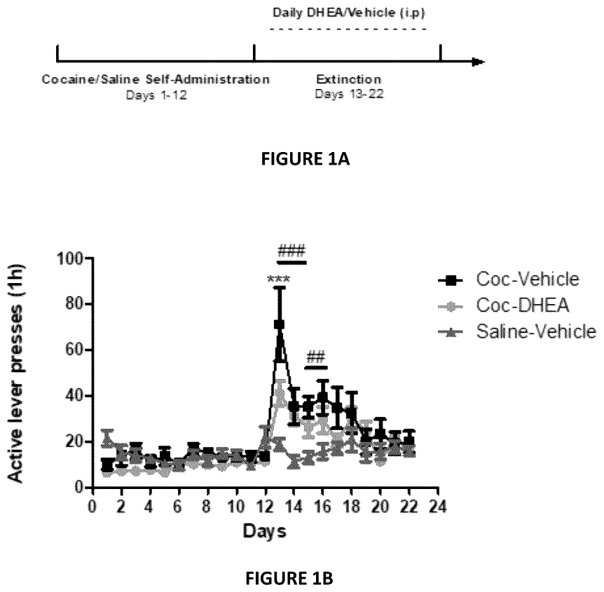

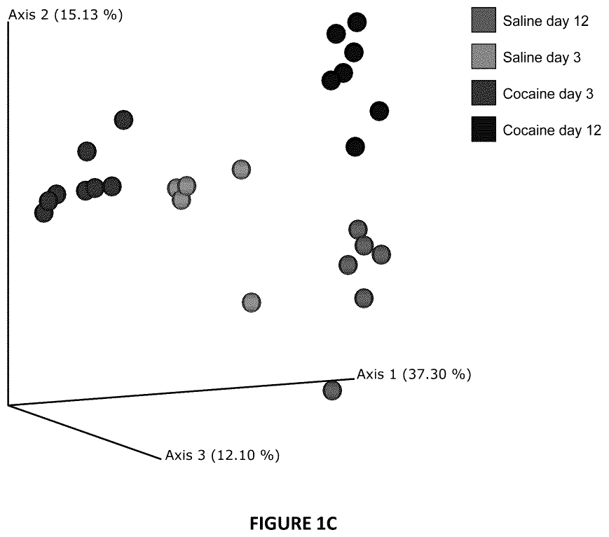

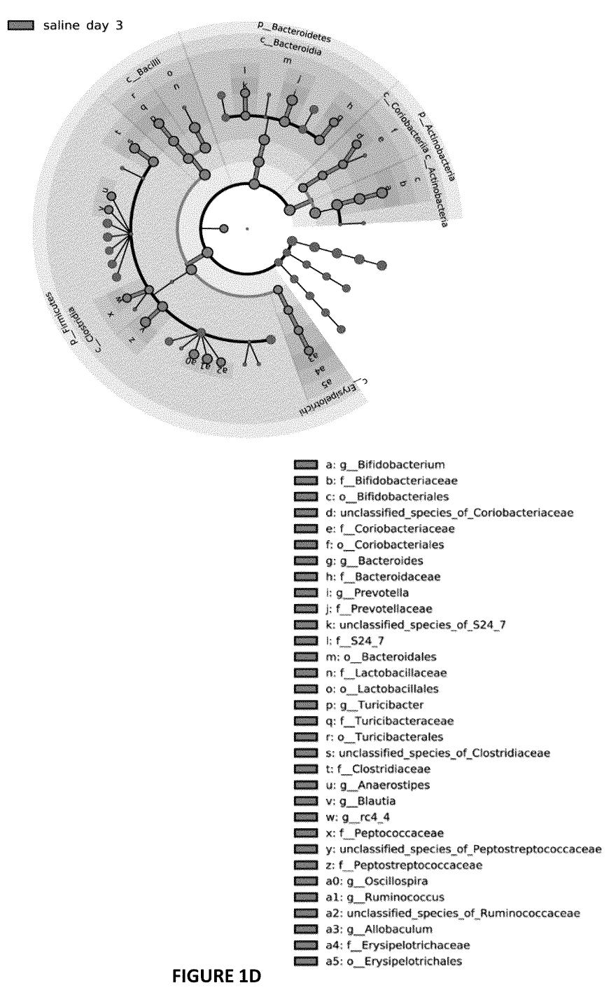

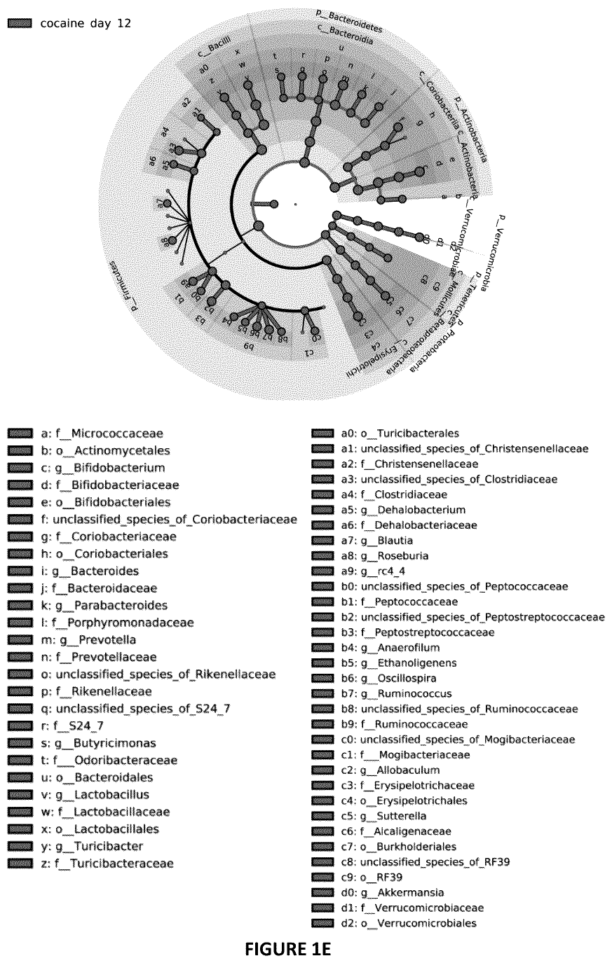

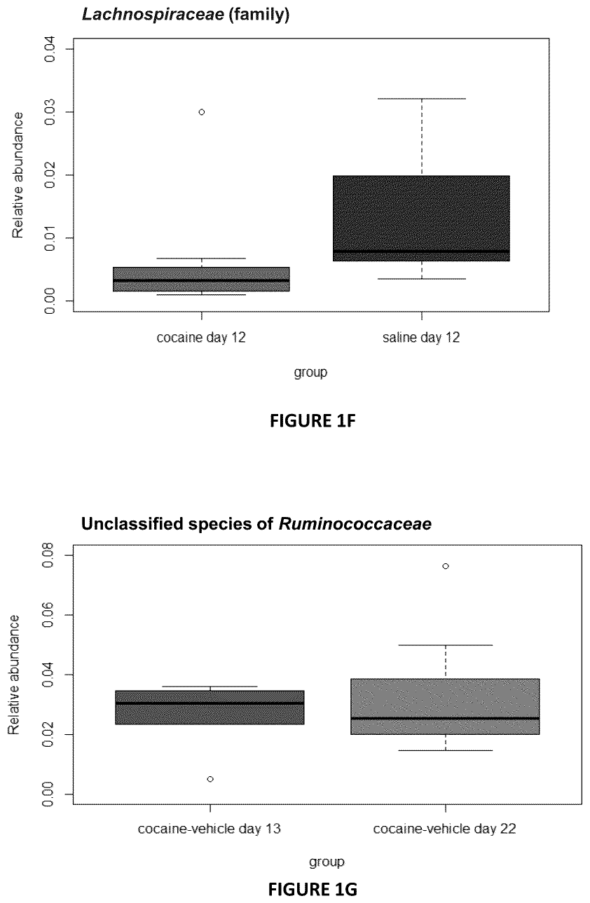

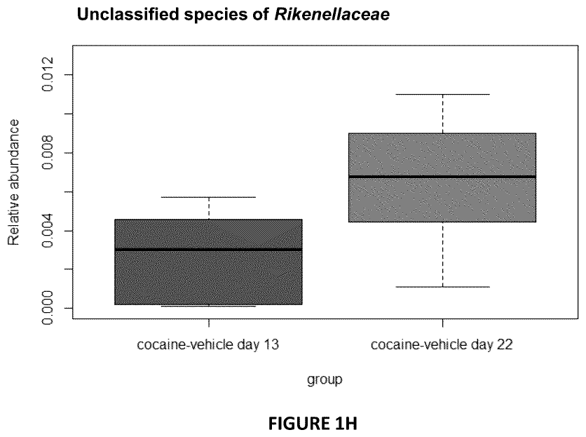

Rats were trained for cocaine or saline self-administration until maintenance. Next, animals were divided randomly into three groups (n=8-9 per group): (1) Saline-Vehicle-rats trained for saline self-administration and treated through extinction with saline, (2) Coc-Vehicle-rats trained for cocaine self-administration treated through extinction with saline, and (3) Coc-DHEA-rats trained for cocaine self-administration and treated through extinction with DHEA (FIG. 1). Another set of rats were trained to self-administer cocaine or saline as described. Upon reaching the maintenance phase, these rats were divided as follow (n=8-9 per each group): (1) Coc-Vehicle, which were trained for cocaine self-administration and treated with gavage containing PBS twice (day 11 and 16) during extinction, (2) Coc-R. gnavus, which were trained for cocaine self-administration and treated with gavage containing R. gnavus twice (day 11 and 16) during extinction, (3) Saline-Vehicle, which were trained for saline self-administration and treated with gavage containing PBS (day 11 and 16) during extinction, and (4) Saline-R. gnavus, which were trained for saline self-administration and treated with gavage containing R. gnavus twice (day 11 and 16), during extinction.

### Statistical Analysis

The behavioural data are expressed as mean±SEM. One-way Analysis of variance (ANOVA) or two-way ANOVA with repeated measures (days) followed by Bonferroni or Student-Newman-Keuls post-hoc tests were used where appropriate. The microbiota data analysis was performed using QIIME2. Single end sequences were joined; sequence reads were demultiplexed by per-sample barcodes and Illumina-sequenced amplicon read errors were corrected with the Divisive Amplicon Denoising Algorithm (DADA2). A phylogenetic tree was generated using the Greengenes database. All analyses of rat fecal samples were calculated based on a feature table containing features observed in at least 50% samples in each group and a minimum count of 10,000 sequences, and beta diversity was analyzed using unweighted and weighted UniFrac distances and pairwise PERMANOVA tests with an FDR correction. Linear discriminant analysis (LDA) effect size (LEfSe) was also performed to identify which microbial features differed between groups. ANCOM, analysis of composition of microbiomes, was performed and only the taxa with a significance of p<0.05 are presented.

### Example 1

### Cocaine Consumption Alters Gut Microbiota Over Time, and DHEA's Effects on Cocaine Extinction and Craving

Rats demonstrated reliable cocaine self-administration behavior during the 12 daily 1-hour training sessions. The control (Saline-Vehicle) group received saline in the same self-administration setup (FIG. 1A). From day 13, rats received daily injections of vehicle or DHEA (IP), followed by daily extinction sessions (days 13-22). Active lever presses were significantly higher in cocaine-trained rats (Coc-Vehicle) compared to the saline-trained group (Saline-Vehicle) and compared to rats treated with DHEA (FIG. 1B).

Next, the inventors characterized the changes in the gut microbiota throughout the self-administration model, and between the rats trained to self-administer cocaine compared to controls. Cocaine-trained rats showed a significant change in their gut microbial community (beta diversity) throughout cocaine self-administration (day 3 vs day 12, p=0.001, FIG. 1C). LEfSe analysis revealed bacterial taxa which were significantly more abundant at day 3 vs day 12 in saline-trained rats (FIG. 1D). When comparing the bacterial taxa on day 3 to day 12 in the cocaine-trained rats, it was found that certain bacterial taxa were more abundant on day 12 (FIG. 1E). When comparing the microbiota of the cocaine and saline-trained rats on day 12 (FIG. 1F) using ANCOM, the control (saline-vehicle) group showed an increased abundance of the bacterial family Lachnospiraceae. Comparing the gut microbiota of the cocaine-trained rats on days 13 to 22 (the first vs last day of the extinction phase) with ANCOM, an increase in the levels of the Ruminococcaceae and Rikenellaceae bacterial families was observed at the end of the extinction phase (FIGS. 1G-1H).

The inventors next examined whether DHEA influences the composition of fecal bacteria. Previous work demonstrated that chronic exposure to exogenous DHEA (2 mg/kg) attenuated cocaine self-administration and decreased the cocaine seeking behavior of the rats to <20% of their maintenance levels. This data is consistent with the current results, showing that active lever presses were significantly reduced in DHEA-treated, cocaine-trained rats compared to vehicle-treated, cocaine-trained rats (FIG. 1B).

### Example 2

### Administration of DHEA Induces a Shift Towards Gut Microbial Community of a Control Group

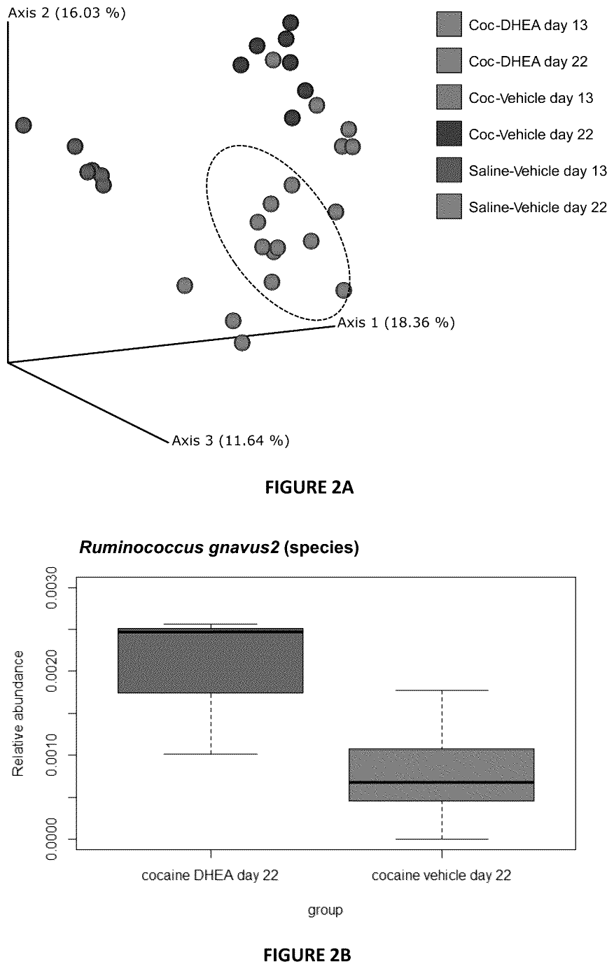

Next the inventors assessed how the gut microbiota composition changes as a result of DHEA delivery during the extinction and withdrawal phases. Remarkably, a PCoA of unweighted UniFrac distances revealed that at the end of the extinction phase (day 22), the gut microbiota of rats that received DHEA (coc-DHEA) resembled those of rats that received vehicle (saline-vehicle) (FIG. 2A). Surprisingly, ANCOM analysis demonstrated an increase in the relative abundance of R. gnavus in cocaine-trained rats treated with DHEA, at the end of extinction and withdrawal phases (FIG. 2B) compared to cocaine-vehicle-trained rats on day 22. These results indicate that DHEA attenuates cocaine seeking behavior, at least in part, by regulating the microbes in the gut and perhaps specifically through increases in R. gnavus.

### Example 3

### R. Gnavus Reduces Cocaine Craving

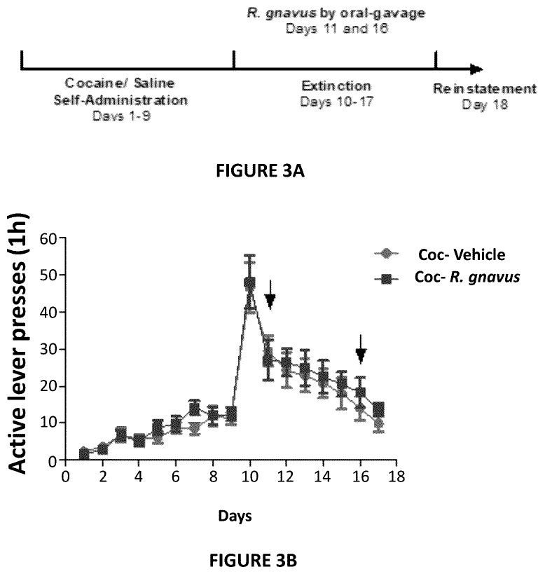

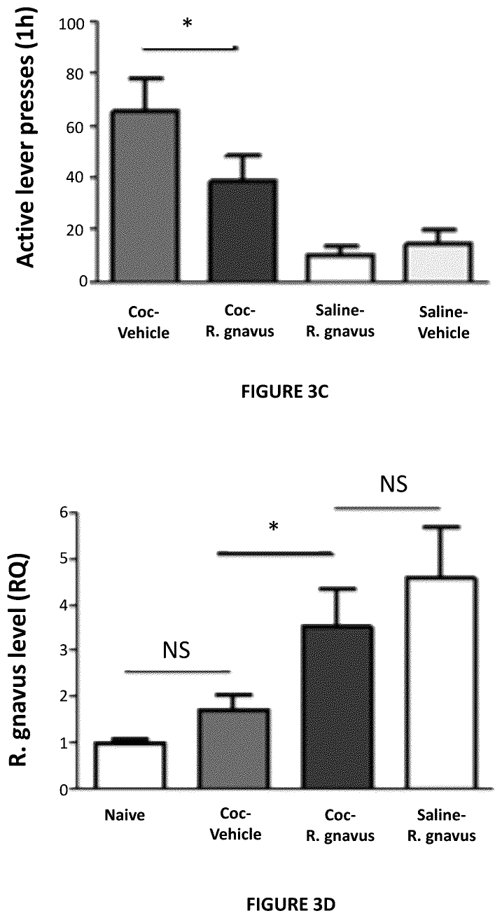

Next, the inventors examined whether R. gnavus plays a role in the behavioral responses to cocaine. Another group of rats was trained to self-administer cocaine (FIG. 3A-3B). Treatment with R. gnavus at the beginning and the end of the withdrawal phases (FIG. 3B) led to a decrease in active lever presses in the reinstatement test as compared with controls (coc-vehicle, FIG. 3C). Thus, treatment with R. gnavus attenuated cocaine craving. In addition, R. gnavus presence was confirmed by qPCR. Rats applied by gavage administration with R. gnavus demonstrated an increase in the abundance of R. gnavus 48 hours after the last gavage administration (FIG. 3D, p<0.05).

### Example 4

### Effect of Cocaine and DHEA on Bacterial Richness and Diversity In Vitro

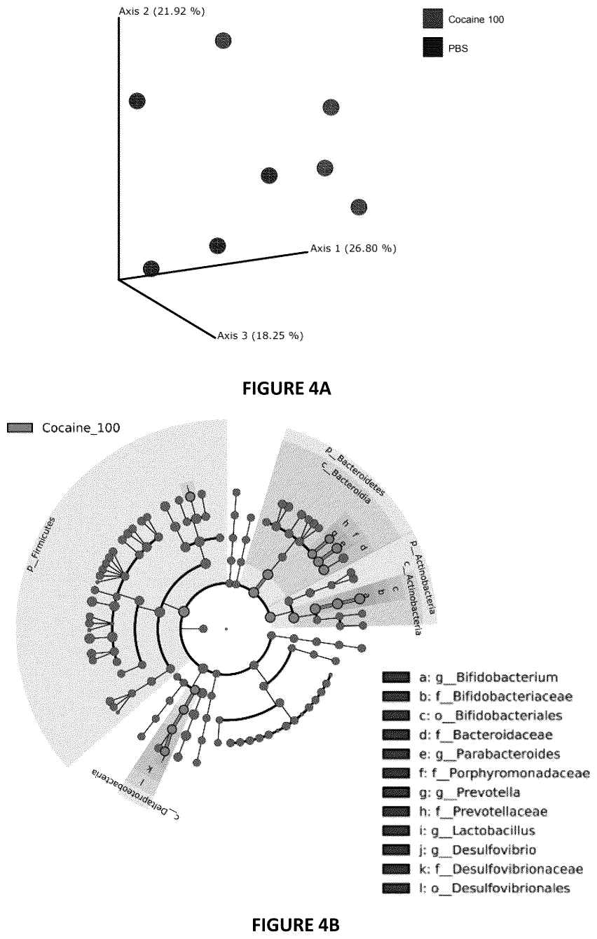

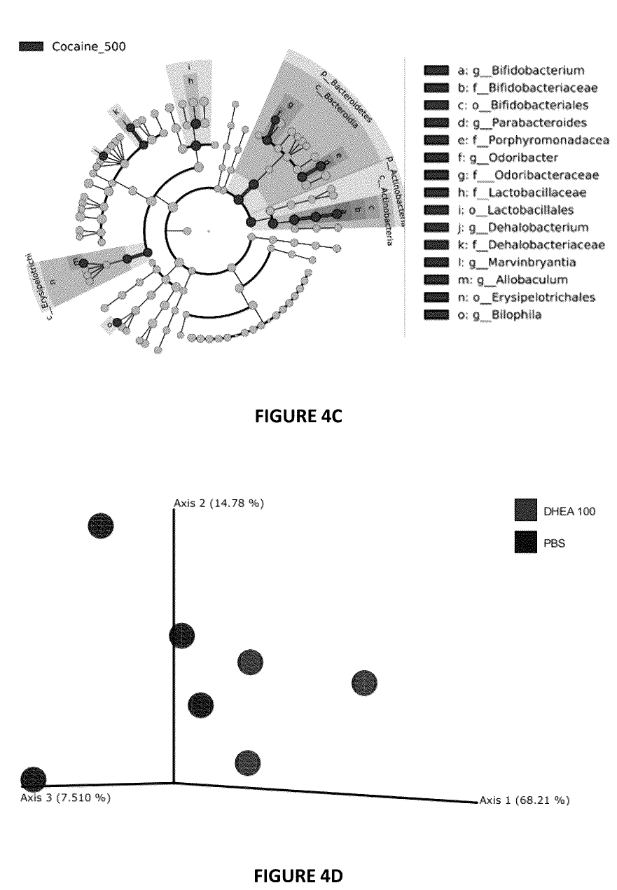

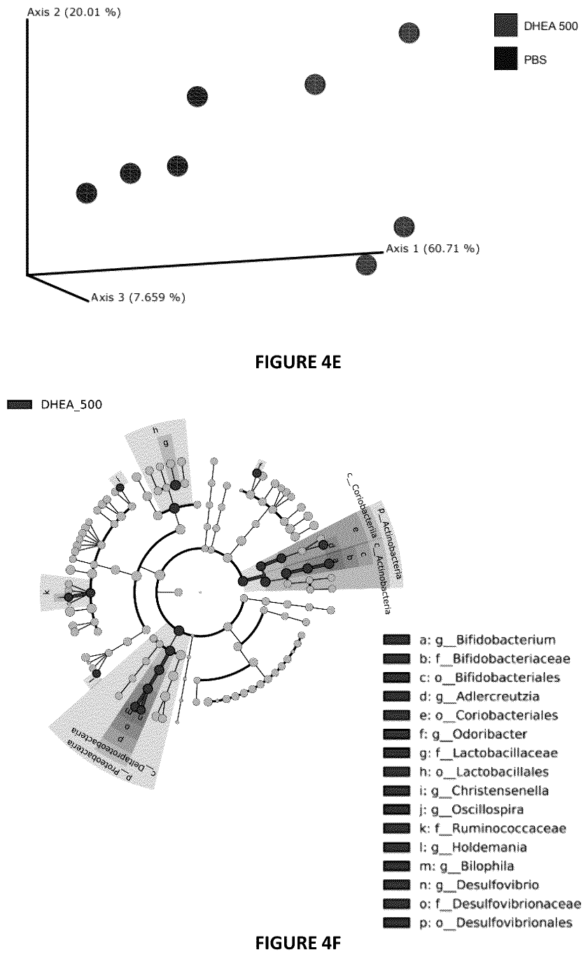

Cocaine at a concentration of 100 μM significantly changed the gut microbiota of naïve rat feces in vitro compared to control feces (PBS), as determined by unweighted UniFrac distances (beta diversity) (FIG. 4A). LEfSe analysis demonstrated an overrepresentation in 12 and 15 bacterial features in response to 100 μM and 500 μM cocaine, respectively, compared to the controls (PBS) (FIGS. 4B-4C). Five of them were similar in both doses (in vitro). Sixteen bacterial features were identical in both in vivo (day 12) and in vitro experiments (both doses), but 43 other bacterial features were found only in feces taken from rats trained to self-administer cocaine (at maintenance, day 12). The inventors also tested the direct effect of DHEA (100 and 500 μM) on feces of naïve rats and found a significant change in the gut microbiota composition compared to controls (PBS) (FIGS. 4D-4E). There were no significant changes in bacterial features as a result of incubation with 50 μM and 100 μM DHEA. However, at 500 μM DHEA, 16 bacterial features were overrepresented (FIG. 4F), though not R. gnavus, as in the in vivo experiment. This finding indicates that the effect of DHEA on R. gnavus in the gut is indirect, suggesting a synergistic effect for combination therapy with DHEA and R. gnavus.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method of treating a substance-use disorder (SUD) in a subject in need thereof, the method comprising administering to said subject a therapeutically effective amount of a pharmaceutical composition comprising bacteria, wherein at least 50% of said bacteria belong to a bacterial family being any one of: Ruminococcaceae, Rikenellaceae, and both, thereby treating SUD in the subject.

2. The method of claim 1, wherein said bacterial family is Ruminococcaceae.

3. The method of claim 1, wherein said bacteria belong to the genus Ruminococcus, and optionally wherein said bacteria belonging to the genus Ruminococcus is Ruminococcus gnavus.

4. (canceled)

5. A method of treating SUD in a subject in need thereof, the method comprising administering to said subject a therapeutically effective amount of a pharmaceutical composition comprising Ruminococcus gnavus bacteria, thereby treating SUD in the subject.

6. The method of claim 1, wherein said subject is afflicted with a severe form of said SUD, and optionally wherein said severe form of said SUD comprises an addiction to said substance.

7. (canceled)

8. The method of claim 1, wherein said SUD is a stimulant-related disorder.

9. The method of claim 8, wherein said stimulant is selected from the group consisting of: cocaine, amphetamine, cathinone, 3,4-methylenedioxymethamphetamine (MDMA), nicotine, any derivative thereof, and any combination thereof.

10. The method of claim 8, wherein said stimulant-related disorder is a cocaine-related disorder.

11. The method of claim 1, wherein said subject is addicted to cocaine or rehabilitating therefrom.

12. The method of claim 1, wherein said effective amount of said pharmaceutical composition comprising said bacteria in an amount of at least 1×10⁴ colony forming unit (CFU).

13. The method of claim 1, wherein said administering comprises orally administering.

14. The method of claim 1, wherein said administering comprises at least once a week administering.

15. The method of claim 1, wherein said treating comprises lowering craving of said subject to said substance.

16. The method of claim 1, wherein said treating comprises increasing an amount of said bacteria belonging to a bacterial family being any one of: Ruminococcaceae, Rikenellaceae, and both by at least 50% in said subject, compared to a control.

17. The method of claim 16, wherein said increased amount of said bacteria belonging to a bacterial family being any one of: Ruminococcaceae, Rikenellaceae, and both, is determined in a sample obtained or derived from said administered subject.

18. The method of claim 1, further comprising a step preceding said administering step, comprising determining an amount of bacteria belonging to a bacterial family being any one of: Ruminococcaceae, Rikenellaceae, and both, in a sample obtained or derived from said administered subject.

19. The method of claim 1, wherein said treating comprises increasing an amount of Ruminococcus gnavus bacteria in said subject, compared to a control, and optionally wherein said increased amount of said Ruminococcus gnavus bacteria is determined in a sample obtained or derived from said administered subject.

20. (canceled)

21. The method of claim 19, further comprising a step preceding said administering step, comprising determining an amount of said Ruminococcus gnavus bacteria in a sample obtained or derived from said administered subject.

22. The method of claim 1, further comprising administering to said subject an effective amount of dehydroepiandrosterone (DHEA), a functional analogue thereof, or a composition comprising thereof, and optionally wherein said effective amount comprises between 0.1 mg/kg (body weight) to 5 mg/kg (body weight) of any one of DHEA and said analogue thereof.

23. (canceled)

24. The method of claim 22, wherein administering said DHEA comprises oral administration, intravenously administering, intraperitoneal administering, or any combination thereof.

25.-31. (canceled)


SEQUENCE LISTING (6 sequences)

SEQ ID NO: 1 - DNA, 32 bp, synthetic construct
  source 1..32: mol_type = other DNA
aatgatacggcgaccaccgagatctacacgct

SEQ ID NO: 2 - DNA, 31 bp, synthetic construct
  source 1..31: mol_type = other DNA
tatggtaattgtgtgycagcmgccgcggtaa

SEQ ID NO: 3 - DNA, 20 bp, synthetic construct
  source 1..20: mol_type = other DNA
acagggacaacggcaaaaga

SEQ ID NO: 4 - DNA, 20 bp, synthetic construct
  source 1..20: mol_type = other DNA
gccattccagctacccagtt

SEQ ID NO: 5 - DNA, 20 bp, synthetic construct
  source 1..20: mol_type = other DNA
actcctacgggaggcagcag

SEQ ID NO: 6 - DNA, 17 bp, synthetic construct
  source 1..17: mol_type = other DNA
attaccgcggctgctgg